(19) United States
(12) Patent Application Publication
(10) Pub. No.: US 2024/0180884 A1
(43) Pub. Date: Jun. 6, 2024

(54) PYRIDOSTIGMINE FOR USE IN THE TREATMENT OF COVID-19

(71) Applicant: INSTITUTO NACIONAL DE CIENCIAS MEDICAS Y NUTRICION SALVADOR ZUBIRAN, Mexico (MX)

(72) Inventor: Sergio Ivan VALDES FERRER, Mexico City (MX)

(21) Appl. No.: 18/286,970

(22) PCT Filed: Apr. 13, 2021
(86) PCT No.: PCT/IB2021/053043

§ 371 Date: Oct. 13, 2023

(51) Int. Cl. A61K 31/4425; A61P 31/14

(52) U.S. Cl. CPC A61K 31/4425; A61P 31/14

(57) ABSTRACT

The present invention relates to the use of pyridostigmine or a pharmaceutically acceptable salt thereof for the treatment of COVID-19.

### FIELD OF THE INVENTION

The present invention is related to the treatment of SARS-COV-2 infection and more particularly is related to the use of pyridostigmine for the treatment of COVID-19.

### BACKGROUND OF THE INVENTION

Infection with the SARS-COV-2 virus causes COVID-19 disease, which can lead to a severe systemic inflammatory response and even death. Approximately one-third of hospitalized COVID-19 patients develop acute respiratory distress syndrome (ARDS) [1]. Systemic inflammation leads to refractory hypoxemia and multi-organic, resulting in increased mortality [2].

Currently, the treatments used to control COVID-19 disease have several shortcomings, including limited effectiveness, high cost, limited availability, need to be administered for long periods of time, and variable efficiency among different patients and given at different points in time. The list of drugs that have been approved by the United States Food and Drug Administration (FDA) for the treatment of COVID-19 disease include the corticosteroid Dexamethasone, and the antiviral drug remdesivir (Veklury™) to treat adults and children 12 years and older who are admitted to the hospital. The FDA has also authorized the emergency use of the rheumatoid arthritis drug baricitinib (Olumiant™) to treat COVID-19 in some cases. Baricitinib is a pill that seems to have an effect against COVID-19 reducing inflammation, and with antiviral activity. The FDA states that baricitinib can be used in combination with remdesivir in people hospitalized with COVID-19 who are undergoing mechanical ventilation or are in need of supplemental oxygen. However, the available treatments to control COVID-19 are still limited, and there is even a great demand for the approved drugs, which generates their shortage and affects people without COVID-19 who regularly need these drugs for other indications. Hospitalized patients with severe COVID-19 can develop a severe systemic inflammatory response, lung damage, and acute respiratory distress syndrome (ARDS), resulting in multi-organ damage and increased mortality.

Pyridostigmine is an inhibitor of the enzyme acetylcholinesterase (i-ACh-e); its use increases the half-life of acetylcholine (ACh) by inhibiting its peripheral degradation. Acetylcholine (ACh) modulates the acute inflammatory response, a neuro-immune mechanism known as the inflammatory reflex. Electrical and chemical stimulation of the inflammatory reflex reduces the load of inflammation in some chronic inflammatory diseases. Pyridostigmine is used for the symptomatic treatment of myasthenia gravis [4, 5] and as pre-exposure prophylaxis for nerve gas poisoning [6]. Pyridostigmine reduces inflammation in chronic human immunodeficiency virus (HIV)-1 infection [7,8,9].

As of today, just over a year after the start of the COVID-19 pandemic, the availability of effective, safe, and inexpensive treatments directed against the inflammation is limited. Therefore, finding new effective immuno-modulating therapeutic routes with an adequate risk/benefit profile is crucial. The World Health Organization (WHO) Solidarity study, for example, has evaluated various treatments for infection. However, antiviral and immunomodulatory therapies against SARS-COV-2 have not generated new therapeutic options of proven utility beyond dexamethasone [10]. Between 25 and 33% of patients hospitalized for COVID-19 require hospitalization in intensive care units (ICU) for severe refractory hypoxemia. Some initial reports from China showed death rates of up to 49% for hospitalized patients with severe COVID-19; however, current estimates place the figure at around 25-27% [11-13].

The severity and mortality of COVID-19 are mediated by the systemic inflammatory response rather than by viremia. Therefore, finding new immunomodulatory strategies is a promising approach to reduce the severity and mortality of COVID-19. Furthermore, repurposing drugs with well-characterized safety profiles and readily available production lines could lead to a faster development of anti-COVID-19 therapies if their efficacy is demonstrated in controlled clinical trials such as the present one. The central nervous system has mechanisms to control the inflammatory response; it has been shown experimentally that in response to an inflammatory insult, the vagus nerve can inhibit the synthesis and release of inflammatory cytokines [14], thus reducing both local damage and mortality secondary to severe systemic inflammation in experimental animal models as diverse as sepsis, ischemia, and reperfusion injury or obesity [15, 16, 17, 18]. The evidence in humans with ARDS and other types of severe inflammatory response has, to the best of our knowledge, not been evaluated before.

The inflammatory response results in release of ACh by the vagus nerve, resulting in reduced production and release of inflammatory mediators, a phenomenon known as the inflammatory reflex. The vagus nerve can be stimulated electrically and chemically. Chemical stimulation with cholinergic agonists has shown promising effects in murine and cellular models of inflammation [18, 19].

Acetylcholine esterase inhibitors (i-ACh-e) are a family of drugs commonly used by thousands of people, including older adults with Alzheimer's disease and other dementias, as well as in patients with myasthenia gravis and dysautonomia [5, 20-23]. These drugs inhibit the enzymatic degradation of endogenous ACh, resulting in increased bioavailability and, therefore, an increase in the possibility of binding to nicotinic or muscarinic type receptors. In addition to the approved uses of i-ACh-e in human pathology, there is evidence in several animal models of its efficacy in experimental polymicrobial sepsis [16, 18, 19], suggesting that i-ACh-e drugs have an Potential immunomodulator in patients with severe systemic inflammatory response syndrome. Pyridostigmine, is an which acetylcholinesterase inhibitor, has previously been shown to reduce aberrant activation and proliferation of T lymphocytes in people living with human immunodeficiency virus (HIV) infection [7-9].

Currently there are some treatments to control COVID-19 disease. For example, the document IN202011016345A relates to a method for treating SARS-COV-2 which comprises administering to the subject a therapeutically effective amount of an ACE2 inhibitor. A therapeutically effective amount of an ACE2 inhibitor is administered in addition to at least one additional agent, such as antiviral agents and glucocorticoids, for the treatment of acute SARS-COV-2 infection. However, the ACE2 inhibitor blocks the degradation of bradykinin, which causes the levels of this protein to increase and the blood vessels to widen, generating vasodilation in patients, which generates a dry cough and could exacerbate the symptoms of COVID-19.

The document IN202043015189 refers to a composition that comprises doxycycline and ribavirin to treat infections including COVID-19, SARS, Ebola, hepatitis B virus, and hepatitis C virus. However, the use of ribavirin has common contraindications such as nausea, vomiting, diarrhea, and difficulty breathing, among others.

As a consequence of the above, it has been sought to find effective treatments for COVID-19 with, at the same time, a safe adverse-event profile than those of treatments for currently used for COVID-19. That resulted in the use of pyridostigmine for the treatment of COVID-19, thus seeking alternatives that offer availability to the population, with a multi-year pharmacovigilance, which improves the response of the immune system of patients and thereby reduces the need for invasive mechanical ventilation and therefore mortality in hospitalized patients with COVID-19 in serious condition.

### OBJECTS OF THE INVENTION

Taking into account the shortcomings of the prior technique, an object of the present invention is the use of pyridostigmine for the treatment of COVID-19 that allows to improve the severity of symptoms produced by said disease.

Another object of the present invention is the use of pyridostigmine for the treatment of COVID-19 that allows for reducing mortality in hospitalized patients with severe COVID-19.

Finally, another object of the present invention is that the use of pyridostigmine for the treatment of COVID-19 is safe, effective, accessible to the population and with a pharmacovigilance of several years.

These and other objects are achieved through the use of pyridostigmine for the treatment of COVID-19 in accordance with the present invention.

### BRIEF DESCRIPTION OF THE INVENTION

One aspect of the present invention is the use of pyridostigmine or a pharmaceutically acceptable salt thereof for the treatment of COVID-19.

### BRIEF DESCRIPTION OF THE FIGURES

The novel aspects that are considered characteristic of the present invention will be set forth focused in the appended claims. However, some modalities, characteristics and some objects and advantages thereof, will be better understood in the detailed description, when read in connection with the attached drawings, in which:

FIG. 1 shows a snapshot of the recruitment and randomization of patients in accordance with the present invention.

FIG. 2 shows the percentage of patients who underwent invasive mechanical ventilation in accordance with the present invention.

FIG. 3 shows the probability of a patient being discharged from the hospital within 28 days of randomization in accordance with the present invention.

### DETAILED DESCRIPTION OF THE INVENTION

The present invention presents certain advantages over the state of the art, among which it can be mentioned that the use of pyridostigmine for the treatment of COVID-19 makes it possible to improve the symptoms produced by said disease, reduces the need for a composite outcome including invasive mechanical ventilation and mortality in hospitalized patients with severe COVID-19. In addition, the use of pyridostigmine is safe, effective and accessible to the population.

Therefore, it is an aspect of the present invention to use pyridostigmine or a pharmaceutically salt thereof for the treatment of COVID-19.

In a preferred embodiment of the present invention, the administration dose of pyridostigmine or a pharmaceutically acceptable salt thereof for a patient is 60 mg/day. Preferably, the patient is of legal age. Preferably, pyridostigmine or a pharmaceutically acceptable salt thereof is administered for a maximum period of 14 days. Preferably, pyridostigmine or a pharmaceutically acceptable salt thereof is administered orally.

Another aspect of the present invention is pyridostigmine or a pharmaceutically acceptable salt thereof for use in treating COVID-19.

It should be noted that the use of pyridostigmine for the treatment of COVID-19 allows to reduce the inflammatory response is regulated by the central nervous system through the release of acetylcholine through via the vagus nerve, resulting in a reduction in the production and release of inflammatory mediators, therefore, pyridostigmine presumes an effect that is reflected as a decrease in the production of pro-inflammatory cytokines in COVID-19 patients at high risk of severe disease.

The present invention will be better understood from the following examples, which are presented for illustrative purposes only to allow a thorough understanding of the preferred embodiments of the present invention, without implying that there are no other, non-illustrated embodiments that can be carried out. the practice based on the detailed description made above.

### Example 1

A trial was conducted to evaluate in a controlled clinical trial setting the efficacy of the use of pyridostigmine as an immuno-modulator in patients with COVID-19 in serious condition in accordance with the present invention.

The trial was conducted to reduce the incidence of mechanical ventilation or death in adults hospitalized due to severe COVID-19. A randomized, parallel, double-blind, placebo-controlled clinical trial was conducted. Participants were randomized (1:1 ratio) to receive oral pyridostigmine at a dose of 60 mg/day orally or an identical placebo (drug-grade starch) until the occurrence of any of the a priori specified outcomes, the hospital discharge or a maximum period of 14 days of hospital confinement, whichever comes first. The study was externally and independently watched and monitored by an independent Data and Security Monitoring Committee (DSMB). The study was conducted in two parts: an initial phase aimed at determining safety (from May 5 to Jul. 4, 2020) in which the first 44 participants were evaluated, followed by a part of a phase aimed at evaluating the effect, or lack thereof, of pyridostigmine in patients with severe COVID-19 (Jul. 5, 2020 to Jan. 30, 2021).

Adult hospitalized patients (>18 years) with confirmed SARS-COV-2 infection based on a positive RT-PCR test for SARS-COV-2 ribonucleic acid in a respiratory sample (nasopharyngeal or nasal swab) and a imaging study compatible with pneumonia, and at least one criterion of high risk of death. Table 1 shows the inclusion and exclusion criteria of the study.

TABLE 1

| INCLUSION CRITERIA | | EXCLUSION CRITERIA | | |
|---|---|---|---|---|
| 1) | Adults (≥18 years) | 1) | Allergy to pyridostigmine | |
| 2) | Understanding and signing of consent | 2) | Pregnancy or breastfeeding (women) | |
| | informed | 3) | Critical illness criteria after signature of | |
| 3) | Confirmatory test of infection by SARS- | | the informed consent and before the | |
| | CoV-2 by PCR | | administration of the medication, | |
| 4) | Pneumonia confirmed by imaging | | including: | |
| | studies | | a. | Need for mechanical ventilation |
| 5) | Need for hospitalization with one or | | b. | Admission to ICU for any reason |
| | more of | | c. | Criteria for septic shock or |
| 6) | The following severity criteria: | | | sepsis |

| | a) | Dyspnea | 4) | Concomitant autoimmune disease |
|---|---|---|---|---|
| | b) | Pulmonary infiltrates by | 5) | Known immunodeficiency (including |
| | | tomography >50% of the | | HIV) |
| | | parenchyma | 6) | Need for mechanical ventilation before |
| | c) | Ratio of partial pressure of arterial | | signing the consent or administration of |
| | | oxygen (PaO2) to fraction of | | the first dose of experimental drug |
| | | inspired oxygen (FiO2) <300 mmHg | 7) | Inability of oral or enteral |
| | d) | Any of the following: a) pulse | | administration |
| | | oximetry <90% in ambient air, b) 3% | 8) | Use of immunosuppressants or |
| | | decrease in baseline oximetry, c) | | immunomodulators in the preceding 28 |
| | | need for supplemental oxygen due | | days, including chemotherapy and |
| | | to hypoxemia, or d) need for | | steroids unless they were part of the |
| | | supplemental oxygen according to | | management of COVID-19 established |
| | | medical criteria | | by the medical group |
| 7) | Alteration of one or more of the | | 9) | Participation in interevention clinical |
| | following laboratory parameters: | | | trials in the preceding 28 days |
| | a) | D-dimer >1 ug/mL | | |
| | b) | Ferritin >300 ng/mL | | |
| | c) | C-reactive protein >3 mg/L | | |
| | d) | Lactic dehydrogenase >245 U/L | | |
| | e) | Lymphocytes <800 cells/uL | | |
| | f) | Creatinin-kinase >800 IU/L | | |

Participants were randomized 1:1, with parallel allocation. Demographic information was collected from the participants at baseline, including age, sex, presence of comorbidities (including diabetes mellitus, hypertension, obesity, cardiovascular disease, lung disease or other chronic medical conditions). Participants who had already been discharged from the hospital by day 28 were contacted by phone to assess their vital and functional status.

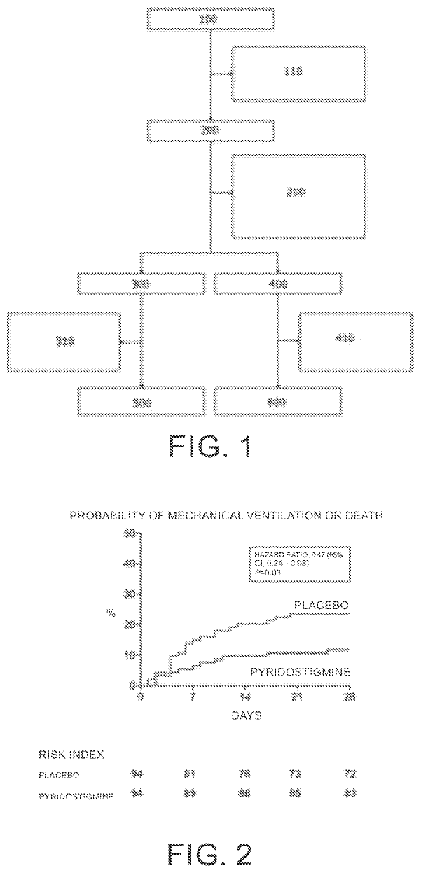

FIG. 1 shows a synoptic table of patient recruitment and randomization, where patients were evaluated for their eligibility (100), of which 98 patients were excluded; 95 of those were excluded due to not meeting inclusion criteria or having exclusion criteria, and 3 were unable to give consent and 35 were eligible, the others refused to participate in the study (110). Then, 201 patients were randomized (200), of which 13 patients were eliminated, 5 withdrew their consent before being randomized or taking the drug, 4 were transferred to another hospital before randomization and before starting the experimental treatment, 3 received mechanical ventilation 15 before randomization, 1 was diagnosed with cancer before receiving experimental treatment (210). Afterwards, of those patients who were eligible, 94 received pyridostigmine (300), of which 7 died before day 28, 7 suspended the drug when transferred to another institution before day 14 and 2 suspended the drug due to adverse effects (310). Of the patients who were eligible, 94 received placebo (400), of which 19 died before day 28, 11 discontinued the drug when transferred to another institution before day 14, and 2 discontinued the drug due to adverse effects (410). Afterwards, patients who received pyridostigmine were included in ‘intention to treat’ on day 28 (500). Similarly the patients who received placebo were included in “intention to treat” on day 28 (600).

Based on early evidence from China, an estimated 25% of patients hospitalized with severe SARS-COV-2 infection would develop complications that would lead to the need for invasive mechanical ventilation or death. Consequently, it is estimated that this sample size would allow us to identify with a power of 80% a reduction in the need for invasive mechanical ventilation or death of 10% in the group that received pyridostigmine compared to the group that received placebo, using a two-tailed Student's t test at the significance level of 0.05. The DSMB performed an interim analysis after the first 44 participants (10% of the calculated sample); after that analysis, DSMB suggested an interim analysis upon reaching 100 participants recruited. Based on the results of said analysis and with the data presented here, it was decided to terminate the study early.

The study was carried out at Instituto Nacional de Ciencias Médicas y Nutrición Salvador Zubirán (principal center) and Instituto Nacional de Cardiología Ignacio Chávez (recruiting center), two COVID-19 designated hospitals in Mexico City, Mexico.

An intention-to-treat analysis was performed comparing the proportion of outcomes between both therapeutic arms using the X² test. A multivariate logistic regression model was used to evaluate the variables associated with primary outcomes.

The principal investigators and the DSMB jointly decided to terminate the trial after recruiting 188 participants after an interim analysis of the first 100 participants carried out on January 26 of this year in which it was observed that the difference between the groups was sufficient to demonstrate a protective effect against the composite outcome including 1) invasive mechanical ventilation (IMV) and 2) death among patients hospitalized for severe COVID-19. At the time of deciding to stop the study, 334 patients had been evaluated and invited, of whom 201 agreed to participate; of these, thirteen patients were excluded for the following reasons: five withdrew consent after signing but before randomization; four were transferred to a different facility before drug administration; three required IMV before drug administration; and one was diagnosed with lung cancer (one of our exclusion criteria) during the initial evaluation and, therefore, these participants were withdrawn from the study before being randomized and did not receive any of the drugs to be evaluated in this study. A total of 188 participants were randomized, randomly assigned to one of the therapeutic arms to evaluate, which are presented in this analysis. Of the participants, 94 received pyridostigmine and 94 received placebo. Seven patients had pyridostigmine treatment suspended before day 14 because they were transferred to another facility and two due to an adverse event or a serious adverse event other than death. Of those assigned to receive placebo, 11 discontinued the placebo before day 14 because they were transferred to another facility and two due to an adverse event or a serious adverse event other than death.

Table 2 shows the baseline demographic and clinical characteristics are balanced between groups.

TABLE 2

| | All | | Pyridostigmine | | Placebo | |
|---|---|---|---|---|---|---|
| Characteristic | (N = 188) | | (N = 94) | | (N = 94) | |
| Sex - no. (%) | | | | | | |
| Male | 112 | (59.6) | 56 | (59.6) | 56 | (59.6) |
| Female | 76 | (40.4) | 38 | (40.4) | 38 | (40.4) |
| Age | | | | | | |
| Mean - yr | 54.8 ± 15.3 | | 53.9 ± 15.1 | | 55.7 ± 15.6 | |
| Median time (IQR) from symptom onset | 10 | (8-12) | 9 | (8-11) | 10 | (8-12) |
| to randomization - days | | | | | | |
| Previous coexisting disease - no. (%) | | | | | | |
| Diabetes | 68 | (36.2) | 33 | (35.1) | 35 | (37.2) |
| Hypertension | 66 | (35.1) | 33 | (35.1) | 33 | (35.1) |
| Heart disease | 4 | (2.1) | 2 | (2.1) | 2 | (2.1) |
| Chronic lung disease | 8 | (4.3) | 6 | (6.4) | 2 | (2.1) |

Mean age of recruited patients was 54.8 years and 59.6% were male. The most common pre-existing conditions were diabetes mellitus and hypertension. There were no significant differences in baseline or pre-exiting conditions between participants.

FIG. 2 shows the percentage of patients who underwent invasive mechanical ventilation or died on day 28 was significantly lower in the pyridostigmine group (11.7%; 95% confidence interval [CI], 6.5 to 19.9 vs. at 23.4%; 95% CI, 15.9 to 32.9; hazard ratio, 0.47; 95% CI, 0.24 to 0.94; p=0.03). By separating the primary outcome into its two components, pyridostigmine resulted in a significant reduction in mortality, including participants who had undergone IMV. Compared to the placebo group, 28-day case fatality was reduced in the pyridostigmine group (7.4%, 95% CI, 3.4 to 14.8 vs. 20.2%, 95% CI, 13.2 to 29.5; risk index, 0.33; 95% CI 0.16 to 0.7; p=0.01).

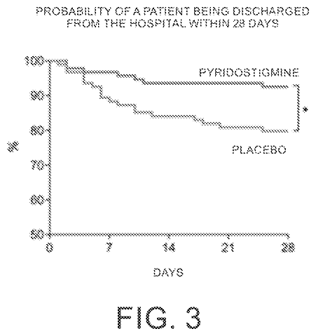

FIG. 3 shows the probability of a patient being discharged from the hospital within 28 days of randomization was higher in patients who received pyridostigmine (92.6%; 95% CI, 85.1 to 96.5 vs. 79.8%; 95% CI, 70.4 to 86.7; hazard ratio, 0.36; CI 95%, 0.16 to 0.79; p=0.01). The use of pyridostigmine did not decrease the probability of undergoing invasive mechanical ventilation (6.3%, 95% CI 2.6 to 13.5 vs 7.4%, 95% CI 3.4 to 14.8). There was no difference between the two groups regarding death after invasive mechanical ventilation (pyridostigmine: 33.3%; 95% CI, 9.2 to 7 vs. placebo: 57.1%; 95% CI, 24.9 to 84.2).

TABLE 3

| | All | | Pyridostigmine | | Placebo | |
|---|---|---|---|---|---|---|
| Characteristic | (N = 188) | | (N = 94) | | (N = 94) | |
| Primary outcome - no. (%) | | | | | | |
| Invasive mechanical ventilation or death | 33 | (17.6) | 11 | (11.7) | 22 | (23.4) |
| Secondary outcomes | | | | | | |
| Invasive mechanical ventilation - no. (%) | 13 | (6.9) | 6 | (6.4) | 7 | (7.5) |
| Death - no. (%) | 26 | (13.8) | 7 | (7.5) | 19 | (20.2) |
| Death after invasive mechanical | 6 | (3.2) | 2 | (2.1) | 4 | (4.3) |
| ventilation - no. (%) | | | | | | |
| Discharge - no. (%) | 162 | (86.2) | 87 | (92.6) | 75 | (79.8) |
| Median time (IQR) from randomization to | 5 | (3.3-8) | 5 | (3-27) | 5 | (4-61) |
| discharge - days | | | | | | |

In the conducted trial, evidence shows that pyridostigmine reduces mortality in patients hospitalized for severe COVID-19. With regard to safety concerns, at the proposed dose of pyridostigmine, the rate of adverse events is less than 5-6% with no serious adverse effects reported. From this perspective, it is considered that pyridostigmine can function as an immunomodulator and reduce morbidity and mortality in these patients. Reducing the frequency of the need for mechanical ventilation would help reduce mortality and the demand for these services.

As can be seen in this trial, the clinical trial carried out allows to verify the efficacy of the use of pyridostigmine as an immuno-modulator in patients with COVID-19 in serious condition.

In accordance with the above data, it can be observed that the use of pyridostigmine for the treatment of COVID-19 has been designed to improve the symptoms produced by this disease, reduce the need for invasive mechanical ventilation and mortality in hospitalized patients with severe COVID-19, and it will be apparent to any person skilled in the art that the modalities of the use of pyridostigmine for the treatment of COVID-19 as described above and illustrated in the accompanying drawings, are only illustrative but not limiting of the present invention, since numerous considerable changes in its details are possible without departing from the scope of the invention.

Therefore, the present invention should not be construed as restricted except as required by the prior art and for the scope of the appended claims.

### REFERENCES

1. Huang C, Wang Y, Li X, Ren L, Zhao J, Hu Y, et al. Clinical features of patients infected 5 with 2019 novel coronavirus in Wuhan, China. Lancet. 2020; 395:497-506.
2. Ruan Q, Yang K, Wang W, Jiang L, Song J. Clinical predictors of mortality due to COVID-19 based on an analysis of data of 150 patients from Wuhan, China. Intensive Care Med. 2020; 46:846-8.
3. Mehta P, McAuley D F, Brown M, Sanchez E, Tattersall R S, Manson J J. COVID-19: 10 consider cytokine storm syndromes and immunosuppression. Lancet. 2020; 6736:19-20. doi:10.1016/S0140-6736(20)30628-0.
4. Andersen J B, Engeland A, Owe J F, Gilhus N E. Myasthenia gravis requiring pyridostigmine treatment in a national population cohort. Eur J Neurol. 2010; 17:1445-50.
5. Gilhus N E, Verschuuren J J. Myasthenia gravis: Subgroup classification and therapeutic strategies. Lancet Neurol. 2015; 14:1023-36. doi:10.1016/S1474-4422(15)00145-3.
6. Keeler J R, Hurst C G, Dunn M A. Pyridostigmine Used as a Nerve Agent Pretreatment Under Wartime Conditions. JAMA J Am Med Assoc. 1991; 266:693-5.
7. Valdés-Ferrer S, Crispin J, Belaunzarán-Zamudio P F, Cantú-Brito C, Sierra-madero J, Alcocer-varela J. Acetylcholine-Esterase Inhibitor Pyridostigmine Decreases T cell overactivation in 20 patients infected by HIV. AIDS Res Hum Retroviruses. 2009; 25.
8. Valdés-Ferrer S I, Crispín J C, Belaunzarán-Zamudio P F, Rodríguez-Osorio C A, Cacho-Diaz B, Alcocer-Varela J, et al. Add-on Pyridostigmine enhances CD4+ T-cell recovery in HIV-1-infected immunological non-responders: A proof-of-concept study. Front Immunol. 2017, 8 Oct.:1-6.
9. Robinson-Papp J, Nmashie A, Pedowitz E, George M C, Sharma S, Murray J, et al. The 25 effect of pyridostigmine on small intestinal bacterial overgrowth (SIBO) and plasma inflammatory biomarkers in HIV-associated autonomic neuropathies. J Neurovirol. 2019; 25:551-9.
10. Repurposed Antiviral Drugs for Covid-19—Interim WHO Solidarity Trial Results. N Engl J Med. 2021; 384:497-511.
11. Olivas-Martínez A, Cárdenas-Fragoso J L, Jiménez J V, Lozano-Cruz O A, Ortiz-Brizuela E, Tovar-Mendez V H, et al. In-hospital mortality from severe COVID-19 in a tertiary care center in Mexico City; causes of death, risk factors and the impact of hospital saturation. PLOS One. 2021; 16:e0245772.
12. Zhou F, Yu T, Du R, Fan G, Liu Y, Liu Z, et al. Clinical course and risk factors for mortality of adult inpatients with COVID-19 in Wuhan, China: a retrospective cohort study. Lancet. 2020; 6736:1-9. doi:10.1016/S0140-6736(20)30566-3.
13. Wu Z, McGoogan J M. Characteristics of and Important Lessons From the Coronavirus Disease 2019 (COVID-19) Outbreak in China: Summary of a Report of 72 314 Cases From the Chinese Center for Disease Control and Prevention. Jama. 2020; 2019:3-6. doi:10.1001/jama.2020.2648.
14. Chavan S S, Tracey K J. Essential Neuroscience in Immunology. J Immunol. 2017; 198:3389-97.
15. Yeboah M M, Xue X, Duan B, Ochani M, Tracey K J, Susin M, et al. Cholinergic agonists attenuate renal ischemia-reperfusion injury in rats. Kidney Int. 2008; 74:62-9. doi:10.1038/ki.2008.94.
16. Satapathy S K, Ochani M, Dancho M, Hudson L K, Rosas-Ballina M, Valdes-Ferrer S I, et al. Galantamine alleviates inflammation and other obesity-associated complications in high-fat diet-fed mice. Mol Med. 2011; 17.
17. Zaghloul N, Addorisio M E, Silverman H A, Patel H L, Valdés-Ferrer S I, Ayasolla K R, et al. Forebrain cholinergic dysfunction and systemic and brain inflammation in murine sepsis survivors. Front Immunol. 2017, 8 Dec.
18. Lehner K R, Silverman H A, Addorisio M E, Roy A, Al-Onaizi M A, Levine Y, et al. 10 Forebrain cholinergic signaling regulates innate immune responses and inflammation. Front Immunol. 2019, 10 Apr.:1-11.
19. Rosas-Ballina M, Valdés-Ferrer S I, Dancho M E, Ochani M, Katz D, Cheng K F, et al. Xanomeline suppresses excessive pro-inflammatory cytokine responses through neural signal-mediated pathways and improves survival in lethal inflammation. Brain Behav Immun. 2015; 44:19-27.
20. Mayeux R. Early Alzheimer's Disease. N Engl J Med. 2010; 362:2194-201. doi:10.1056/NEJMcp0910236.
21. Querfurth H W, LaFerla F M. Alzheimer's Disease. N Engl J Med. 2010; 362:329-44. doi:10.1056/NEJMra0909142.
22. O'Brien J T, Thomas A. Vascular dementia. Lancet. 2015; 386:1698-706. doi:10.1016/S0140-6736(15)00463-8.
23. Singer W, Sandroni P, Opfer-Gehrking T L, Suarez G A, Klein C M, Hines S, et al. Pyridostigmine treatment trial in neurogenic orthostatic hypotension. Arch Neurol. 2006; 63:513-8.

1. The use of pyridostigmine or a pharmaceutically acceptable salt thereof for the treatment of SEVERE COVID-19.

2. The use of pyridostigmine or a pharmaceutically acceptable salt thereof for the treatment of COVID-19 according to claim 1, wherein the administration dose of pyridostigmine or a pharmaceutically acceptable salt thereof for a patient is 60 mg/day.

3. The use of pyridostigmine or a pharmaceutically acceptable salt thereof for the treatment of COVID-19 according to claim 2, where in addition because the patient is of legal age.

4. The use of pyridostigmine or a pharmaceutically acceptable salt thereof for the treatment of COVID-19 according to claim 1, wherein the pyridostigmine or a pharmaceutically acceptable salt thereof is administered for a maximum period of 14 days.

5. The use of pyridostigmine or a pharmaceutically acceptable salt thereof for the treatment of COVID-19 according to claim 1, wherein pyridostigmine or a pharmaceutically acceptable salt thereof is administered orally.

6. Pyridostigmine or a pharmaceutically acceptable salt thereof for use in the treatment of COVID-19.
